(19) United States
(12) Patent Application Publication
(10) Pub. No.: US 2003/0087318 A1
(43) Pub. Date: May 8, 2003

(54) Antibodies against an extracellular matrix complex and their use in the detection of cancer

(76) Inventor: Roger L. Lallone, Birmingham, AL (US)

Correspondence Address: ATTN: Louis C. Cullman, OPPENHEIMER WOLFF & DONNELLY LLP, Suite 700, 840 Newport Center Drive, Newport Beach, CA, 92660, US

(21) Appl. No.: 10/243,097

(22) Filed: Sep. 12, 2002

Related U.S. Application Data
(63) Continuation of application No. 08/325,659, filed on Oct. 18, 1994, now abandoned.
Application No. 08/325,659 is a continuation of application No. 08/229,290, filed on Apr. 18, 1994, now abandoned.

(51) Int. Cl. G01N 33/574; G01N 33/53; G01N 33/537; G01N 33/543

(52) U.S. Cl. 435/7.23; 435/7.93

(57) ABSTRACT

A method for assaying for cancer in a mammal. The method comprises attaching a trapping antibody to a surface, wherein the trapping antibody recognizes extracellular matrix (ECM) complexes. A biological fluid sample is contacted with the trapping antibody attached to the surface to thereby bind any ECM complex present in the biological fluid to the trapping antibody to form bound ECM complex. A signaling antibody is prepared and bound to bound ECM complex, wherein the signaling antibody recognizes ECM complex. The amount of signaling antibody bound to the surface is then quantitated, wherein accumulation of signaling antibody on the surface, above a background accumulation, indicates a positive result for cancer.

This is a continuation of pending patent application Ser. No. 08/229,290, filed Apr. 18, 1994.

### FIELD OF INVENTION

The present invention relates to cancer detection and, more particularly, to a method for determining the presence of a substance in extracellular fluids which can be used to indicate the presence of cancer in humans and animals.

### BACKGROUND OF THE INVENTION

Cancer is a disease characterized by uncontrolled cellular proliferation resulting in invasion and destruction of adjacent tissues and metastases to distant organs. Cancer is often fatal but progress is constantly being made toward developing methods for effecting remission for longer and longer periods and even “cure” of patients with certain cancers. Early diagnosis of cancer or recurrence after remission improves prognosis. Treatment regimes, which include combinations of surgery, chemotherapy, local and whole body irradiation, and more recently, the use of biological response modifiers are often most effective when total tumor burdens are low.

There are 3 steps which are thought to be involved in the process of invasion of a metastatic tumor into new tissue. The first involves the receptor mediated attachment of cancer cells to connective tissues, stromal elements, and basements membranes. The second involves proteolytic digestion of the extracellular matrix of the target tissue. The proteolytic digestion results in the liberation of a wide variety of extracellular matrix proteins such as collagens, fibronectins, thrombospondin, laminin, vitronectin and elastin. The third step involves locomotion into the target tissue and the establishment of a tumor specific extracellular matrix.

Several studies have been direct toward identifying unique forms of extracellular matrix proteins associated with malignant tumor growth. For example, onco-fetal forms of collagen type I, fibronectin, and fibrinogen have been identified and characterized. However, such assays require solid tissue biopsy material and, therefore, are impractical to institute as a routine diagnostic assay.

Other assays have attempted to use non-invasive procedures. One such assay is directed at the development of an immunoassay which detects a collagen type III specific amino-terminal propeptide. The propeptide is cleaved off and released as newly synthesized collagen molecules are processed. The levels of these peptides have been shown to correlate well with disease progression in ovarian cancer and, therefore, to be an indicator of ovarian cancer. However, other conditions, and normal physiological fluctuations, unrelated to cancer resulted in increased levels of the peptide, making the usefulness of this marker limited.

Other strategies have been directed at assays for enzyme induced destruction of extracellular matrix associated with tumor growth. These assays include: 1) direct and indirect measurements of tumor derived collagenase, collagenase production (i.e. specific mRNA), and collagenase activity and 2) measurements of collagen fragments and collagen specific amino acids in serum, plasma, and urine. These methods have been unsuccessful as diagnostic tools either because they require solid tissue biopsy samples from affected tissues or because collagen degradation products can not be identified in serum.

A variety of techniques are currently used for early diagnosis. These techniques have been developed to overcome the inaccuracy of external detection techniques, the invasiveness of surgical biopsy techniques and the expense associated with radiological and ultrasound techniques. These techniques rely on a variety of in vitro assays which detect byproducts of tumor growth in extracellular fluids. The majority of these assays are simple immunoassays which detect antigens, in serum and plasma, that are shed from the tumor cell during cell division and proliferation. As a result the assays are generally specific for certain malignant cell types. For example: the CEA and CA19-9 antigens are associated with cancer of the colon, lung, stomach, and pancreas; the AFP antigen is associated with testicular and liver cancers; the CA 15-3 and HER2/neu antigens are associated with breast cancers; and most recently the PSA and PAP antigens have been shown to be associated with prostate cancer.

Many such antigens or serum markers have been identified and are often used in assays in combination with each other. The routine use of such diagnostic tests is expensive and laborious since each form of cancer requires a separate antigen or serum marker assay. The cost and time involved in such testing precludes routine screening of a large population, a form of testing which would be most effective in the diagnosis of cancer.

It is desirable that a test system is developed which is cost effective and which detects any form of cancer with a single test.

### SUMMARY OF THE INVENTION

The present invention is directed at a method for assaying for cancer in a mammal. The method comprises attaching a trapping antibody to a surface, wherein the trapping antibody recognizes extracellular matrix (ECM) complexes. A biological fluid sample is contacted with the trapping antibody attached to the surface to thereby bind any ECM complex present in the biological fluid to the trapping antibody to form bound ECM complex. A signaling antibody is prepared and bound to bound ECM complex, wherein the signaling antibody recognizes ECM complex. The amount of signaling antibody bound to the surface is then quantitated, wherein accumulation of signaling antibody on the surface, above a background accumulation, indicates a positive result for cancer. In one embodiment of the present invention samples which test positive for the presence of cancer are further analyzed, using anti-collagen antibodies to identify the specific form of cancer involved.

The present invention is also directed at the preparation of such trapping and signal antibodies.

### DETAILED DESCRIPTION

Serum and plasma tests for cancer, which are currently available, are directed at the detection of proteins, lipids, and carbohydrate antigens shed or secreted by specific malignant cell types. The present invention is directed at the detection of aggregates of extracellular matrix (ECM) proteins (the ECM aggregates or complexes) as a pan cancer marker. The pan marker of the present invention comprises connective tissue proteins which are not found in high concentration in the serum of normal patients but which are present, in high concentrations, in the serum of cancer patients. Therefore, assays for the ECM complex of the present invention provide a non-invasive assay suitable for routine screening for cancer. Detection of such connective tissue proteins in serum is contrary to the teachings of the art.

The present invention is also directed at a second assay scheme for identifying the type of tissue involved in a patient who has tested positive with the pap (ECM complex) marker. This second assay is directed at analyzing type specific collagen fragments present in the ECM aggregate.

Eleven distinct collagen types have been identified. The best characterized collagen subtypes include types I, II, III, IV, V and VI. Their distributions vary according to the tissue in which they are found. Of these: Type I collagen is associated with stroma, interstitial space, skin, bone, tendon, ligament, facia, dentin and blood vessels; Type II collagen is associated with cartilage, nucleus pulposus, vitreous body and interstitial space; Type III collagen is associated with stroma, interstitial space, skin, bone, ligament, facia, dentin, blood vessels, gastrointestinal tract and fetal skin; Type IV collagen is associated with basement membrane, kidney, glomeruli and lens capsule; Type V collagen is associated with basement membrane and is widespread in small amounts; and Type VI collagen is associated with basement membrane and is ubiquitous to most mammalian tissues.

### Extracellular Matrix Complex Purification

The ECM aggregate or complex of the present invention comprises a variety of different proteins and protein fragments. The ECM complex is isolated from different individuals and, as a result, differences in the physical properties of the ECM complex are observed. These differences make it necessary to adjust the purification procedure to accommodate the different ECM complexes present in the serum sample. The occurrences of such differences in human and animal tissue is well known to those skilled in the art.

Generally, the process for the purification of ECM complex is from biological fluids of a cancer patient. The biological fluids are collected and processed separately. The fluids are diluted about 1:2 to 1:3 with a buffered saline solution such as phosphate, Tris or borate buffered saline (100 mM buffer, pH 7.4, 137 mM NaCl, 1.6 mM KCl) and filtered through course paper to remove debris. The solution is then clarified by centrifugation at about 10,000 to 20,000×g for about 10 to 30 minutes. The supernatants obtained from the centrifugation are checked visually for clarity and absence of particulate material. If particulate matter remains the supernatants are re-centrifuged at a higher “g” force and/or for a longer time until a “clear” supernatant is obtained.

The most useful method for initial purification of the ECM complexes is sulfate precipitation using a sulfate salt such as ammonium sulfate, sodium sulfate or other suitable sulfate salts. In most cases, but not all, precipitation of the supernatant is achieved at relatively low concentrations of sulfate (e.g. 1 M (NH₄)₂SO₄). The resultant precipitate is collected by sedimentation under gravity or centrifugation at about 10,000 to 20,000×g for about 10 to 30 minutes. However, not all samples precipitate as described above and it may be necessary to increase the sulfate concentration up to, but not exceeding, about 2 M. It is undesirable to use concentrations of sulfate above about 2 M since proteins, other than the ECM complex, will also precipitate.

The sulfate precipitated material is dissolved in a small volume of buffered saline. As used herein “a small volume” is the smallest amount of buffered saline required to solubilize the sulfate precipitate. In some cases solubility of the sulfate precipitate can be increased by dialyzing the resuspended sulfate precipitate into a solution comprising about 0.5 to 1 M NaCl. Particulate matter which does not dissolve or which forms during dialysis is collected by centrifugation and discarded.

In samples where the use of sulfate precipitation fails to precipitate the desired protein complex or where a more highly purified ECM complex is desired, the clarified biological fluid or sulfate precipitate is dialyzed against a solution with a low ionic strength such as deionized or distilled water. The material is dialyzed extensively against several changes of the low ionic strength solution until a precipitate forms. The resultant precipitate is collected by centrifugation at about 10,000 to 20,000×g for about 10 to 30 minutes and redissolved in buffered saline with or without NaCl. A NaCl concentration of about 0.5 to 1 M may be used if desired.

In another embodiment of the present invention clarified biological fluid or partially purified ECM aggregate is dialyzed against a solution comprising about 2 M glycine, or glycine is added to a final concentration of about 2 M. The precipitate which forms is collected by centrifugation at about 10,000 to 20,000×g for about 10 to 30 minutes and redissolved in buffered saline.

In another embodiment of the present invention, which can be used where the methods described above have proven to be ineffective, or where a more highly purified ECM complex preparation is desirable, involves precipitation of clarified biological fluid or partially purified ECM complex with NaCl. The precipitation is achieved by adding NaCl, either as a single step precipitation at a final NaCl concentration of about 5.0 M or by step-wise increasing the salt concentration from about 2 M to about 5 M NaCl. Increments of 2, 3, 4 and 5 M NaCl are suitable for use in the present invention. Precipitates are collected by centrifugation at about 10,000 to 20,000×g for about 10 to 30 minutes and redissolved in buffered saline. The redissolved precipitates are analyzed by SDS-PAGE, with and without reduction by 2-mercaptoethanol (2-ME), by methods described by Weber et al., J. Biol. Chem. 244 4406 (1969), as modified by Laemmli, Nature 277 680 (1970) which are incorporated herein by reference.

The most useful ECM complex preparation for use as antigen for the production of antibodies against the ECM complex in the present invention, are those preparations which appear to contain the least amount of high molecular weight material, i.e. material which forms bands at positions corresponding to intact, non-degraded fibrinogen, a molecular weight of about 400,000. Due to the differences in samples, as noted above, not all preparations precipitate under the conditions described and other methods or combinations of the above described methods may be used. Furthermore, the most useful preparations, as determined by SDS-PAGE, will not always correspond to the same fraction from one preparation to another. Therefore, each preparation must by analyzed by SDS-PAGE or other suitable methods to determine which fractions are to be collected for further use.

In most cases, the purified ECM complex has a high molecular weight and gel-filtration chromatography is useful for removing low molecular weight contaminants which may have co-purified with the ECM complex. The ECM complex is obtained from the void volume of gel filtration media such as SEPHAROSE 4B and 6B (Pharmacia, Upsala Sweden) which are preferred for use in the present invention. However, other gel filtration media, well known in the art, could also be used. When other media is used the elution properties of the ECM complex on the media is evaluated to determine where the ECM complex elutes, by methods well known to those skilled in the art. The isolated ECM complex is loaded onto gel filtration medium such as SEPHAROSE 4B or 6B, packed into columns, which have been equilibrated with a buffered saline solution. The material which elutes in the void volume is collected, pooled, concentrated by ultrafiltration, and analyzed by SDS-PAGE.

### Purification of Collagens

Human collagens are extracted from human placental tissues by limited pepsin digestion. The collagens are then purified and separated into their subtypes by precipitation from both neutral and acidic buffers by incremental increases in NaCl concentration. All steps are carried out at 4° C. Precipitates are allowed to form overnight and precipitated material is collected and soluble material clarified by centrifugation at about 10,000 to 20,000×g for about 10 to 30 minutes. Dialysis steps are carried out for about 12 to 18 hours at 4° C., unless otherwise stated.

Tissues are minced, and then washed in saline; water; 0.5 M acetic acid; and finally in 0.5 M formic acid. The material remaining after the washing steps is mixed with pepsin in an acidic solution such as 0.5 M formic acid. The mixture is incubated at about 4° C. with constant mixing for about 12 to 18 hours. At the end of the incubation the soluble material is collected and filtered to remove undigested material. The filtrate is neutralized by the addition of an alkali solution such as 10 M NaOH, to inactivate the pepsin, and then dialyzed into an acetic acid solution.

The digested material is then fractionated as follows: The digested material is dialyzed against 0.5 M acetic acid, 0.6 to 0.7 M NaCl. The precipitate which forms (the 1st precipitate) is collected by centrifugation. The supernatant (the 1st supernatant) is also collected for further processing.

The 1st precipitate is dissolved in 1 M NaCl, clarified by centrifugation and the supernatant is dialyzed against 0.02 M Tris-HCl, pH 7.5, 0.1% w/v (NH₄)₂SO₄. The solution is then dialyzed against 1 M NaCl and centrifuged to remove Particulate matter. The supernatant is collected and dialyzed against 1.5 M NaCl. The precipitate which forms (the 2nd precipitate) is collected by centrifugation. The supernatant (the 2nd supernatant) is also collected.

The 2nd precipitate is dissolved in 1 M NaCl and the solution is clarified by centrifugation. The resultant supernatant (the 3rd supernatant) is collected and dialyzed against 1.5 M NaCl. The precipitate which forms (the 4th precipitate) is collected by centrifugation. The 4th precipitate which includes type III collagen is dissolved in 0.5 M acetic acid and dialyzed against 0.5 M acetic acid.

Sodium chloride is added to the 2nd supernatant to a final concentration of 2 M. The precipitate which forms is collected by centrifugation and discarded. The resultant supernatant (the 4th supernatant) is brought to 2.5 to 4 M NaCl. The precipitate which forms (the 5th precipitate) and supernatant (the 5th supernatant) are collected by centrifugation. The 5th supernatant includes type II collagen. The 5th precipitate, which includes type I collagen, is resuspended in 0.5 M acetic acid and dialyzed against 0.5 M acetic acid.

The 1st supernatant is dialyzed against 0.5 M acetic acid, 1.2 M NaCl and the precipitate which forms (the 6th precipitate) and the resultant supernatant (the 6th supernatant are collected by centrifugation.

The 6th supernatant is dialyzed against 0.5 M acetic acid, 2 M NaCl. The precipitate which forms (the 7th precipitate), which includes type VI collagen, is collected by centrifugation. The supernatant is discarded.

The 7th precipitate is dissolved in 0.5 M acetic acid and dialyzed against 0.2 M sodium phosphate buffer, pH 9. The precipitate which forms (the 8th precipitate) and the resultant supernatant (the 8th supernatant) are collected by centrifugation. The 8th supernatant includes type IV collagen. The 8th precipitate is dissolved in 1 M NaCl. The precipitate which forms (the 9th precipitate), which includes type V collagen, is collected by centrifugation. The supernatant is discarded.

### Preparation of Normal Serum and Plasma for Use in Antibody Purification

Normal human blood is collected either from volunteer donors or patients with disorders unrelated to malignancy. The blood is collected either without the addition of anticoagulants or with the addition of anticoagulants such as heparin, sodium citrate, or EDTA. Serum or plasma is prepared from the blood by methods well known to those skilled in the art. The serum and/or plasma is centrifuged at about 1,000 to 2,000×g for about 30 to 60 minutes. The supernatant is dialyzed against a buffer such as phosphate buffered saline (PBS; 137 mM NaCl, 1.6 mM KCl, 8.1 mM sodium phosphate, 1.5 mM potassium phosphate) with 5 mM EDTA (PBSE), to remove low molecular weight material such as free amino acids and small peptides which might interfere with the coupling reaction described below, to form normal serum and plasma. The normal serum and plasma are then immobilized on a matrix such as SEPHAROSE, silica beads, activated filtration membranes or other suitable material.

### Preparation of Affinity Chromatography Media

SEPHAROSE 4B (Pharmacia) is washed extensively in water and suspended as a 50% slurry in 0.1 M carbonate buffer, pH 11. Cyanogen bromide (such as that supplied by Kodak, Rochester, N.Y.) is dissolved in N,N dimethylformamide (DMF; such as that supplied by Aldrich, Milwaukee, Wis.; Cat. No. D31,993-7) and added to the SEPHAROSE slurry to a final concentration of about 33 mg/ml.

The activation procedure is carried out on ice, in a fume hood, with constant pH monitoring. The pH is maintained at 11 by the addition of 10 M NaOH until the reaction stops and the pH ceases to drop. The activated SEPHAROSE is washed in a vacuum funnel with cold water and then with cold 0.1 M BBS. The washed-activated SEPHAROSE is then coupled to proteins such as normal serum, and plasma protein, ECM complex or purified collagen are added at a maximum of 10 mg of the desired protein per ml of packed volume of SEPHAROSE. The protein/SEPHAROSE mixture is incubated overnight, or longer, at 4° C. to allow the coupling reaction to go to completion. Alternatively, CNBr-activated SEPHAROSE may be purchased commercially from suppliers such as Sigma Chemical Co of St Louis Mo.

Prior to use, the conjugated SEPHAROSE is blocked by incubation with 1 M glycine. The media may be packed into a column or used in a filter funnel for efficient passage of serum antibodies to be affinity purified or cross absorbed.

### Preparation of Polyclonal Antisera

Animals suitable for use to raise hyper-immune antisera are any of those used routinely for this purpose, such as rabbits, goats, sheep, horses and burro. Animals are injected subcutaneously, intradermally, intraperitonealy, intravenously or even in the foot pads, with or without the use of adjuvants such as complete and incomplete Freund's or any of the MDP based Ribi adjuvants known to those skilled in the art.

Animals are segregated into groups. The size of the group is determined by anticipated antibody yields and anticipated needs. One group is immunized with ECM complex, purified as discussed above. Other groups are immunized with preparations of one of the six collagen subtypes I, II, III IV, V and VI.

Since there is great variability in the collagen subtype content of the ECM aggregate purified from individual patients, another group is immunized with a mixture of ECM complex, supplemented with the purified collagen types I, II, III, IV, V and VI.

Any of the standard immunization protocols commonly used by those skilled in the art are suitable for use in the present invention. Antibody responses and specific titers are monitored by ELISA or other suitable assay procedures and immunization schedules, immunogen composition and dosages are adjusted according to conventional criteria well known to those skilled in the art.

### Purification of Polyclonal Antibodies

Antiserum is collected from the animals and purified by affinity chromatography. Any of several purification protocols can be followed and each will yield a polyclonal antibody preparation specific for a certain epitope or combination of epitopes on the ECM complex.

In one embodiment of the present invention, two antibody preparations are used. One for trapping or binding the ECM complex from a biological fluid to a surface (the trapping antibody). This antibody is purified by affinity purification and cross absorption. The material which binds to the affinity chromatography medium is collected while material which binds to the cross absorption chromatography medium is discarded. The trapping antibody is preferably highly specific for the ECM complex. The high specificity of the antibodies is achieved by purifying the antibodies by affinity chromatography on medium which comprises highly purified proteins known to be present in the ECM complex.

The second antibody used in the assays of the present invention are signaling antibodies. The signaling antibodies are used to detect ECM complex bound to the trapping antibodies. It is preferable that the signalling antibodies are broadly reactive to ensure maximum amplification and to minimize the occurrence of false negatives.

Trapping Antibodies

Serum from immunized animals is collected, pooled and diluted with about an equal volume of a buffer such as 0.1 M borate buffered saline (0.1 M BBS; 100 mM sodium borate, pH 7.4, 137 mM NaCl, 1.6 mM KCl). In one embodiment of the present invention the diluted serum is applied to type-VI-collagen-SEPHAROSE equilibrated with a buffer such as 0.1 M BBS. The medium is then washed, in sequence, with a buffer such as 0.1 M glycine-HCl, pH 3.0, then 0.1 M glycine-HCl, pH 2.0. Antibodies which remain bound to the type-VI-collagen-SEPHAROSE are then eluted with 0.1 M HCl containing 0.1 M NaCl. The 0.1 M glycine-HCl, pH 3.0, 0.1 M glycine-HCl, pH 2.0 and 0.1 M HCl washes are collected, neutralized by the addition of 0.1 M BBS and pooled.

In another embodiment of the present invention the diluted serum is applied to ECM-complex-SEPHAROSE equilibrated with a buffer such as 0.1 M BBS. The medium is then washed, in sequence, with a buffer such as 0.1 M glycine-HCl, pH 3.0, then 0.1 M glycine-HCl, pH 2.0. Antibodies which remain bound to the ECM-complex-SEPHAROSE are then eluted with 0.1 M HCl containing 0.1 M NaCl. The 0.1 M glycine-HCl, pH 3.0, 0.1 M glycine-HCl, pH 2.0 and 0.1 M HCl washes are collected, neutralized by the addition of 0.1 M BBS and pooled. The neutralized antibody fraction is then applied to normal-serum-SEPHAROSE equilibrated with a buffer such as 0.1 M BBS. Unbound material is washed from the normal-serum-SEPHAROSE with a buffer such as 0.1 M BBS and reapplied to the normal-serum-SEPHAROSE until all material which will bind to the normal-serum-SEPHAROSE has been bound. The material which remains unbound is collected. Bound material is eluted from the normal-serum-SEPHAROSE with about 0.1 M HCl and discarded.

The material which remains unbound to the normal-serum-SEPHAROSE is then applied to normal-plasma-SEPHAROSE equilibrated with a buffer such as 0.1 M BBS. Unbound material is washed from the normal-plasma-SEPHAROSE with a buffer such as 0.1 M BBS and reapplied to the normal-plasma-SEPHAROSE until all material which will bind to the normal-plasma-SEPHAROSE has been bound. The material which remains unbound is collected. Bound material is eluted from the normal-plasma-SEPHAROSE with about 0.1 M HCl and discarded.

The unbound material from the normal-plasma-SEPHAROSE is then applied to type-IV-collagen-SEPHAROSE equilibrated with a buffer such as 0.1 M BBS. Unbound material is washed from the type-IV-collagen-SEPHAROSE with a buffer such as 0.1 M BBS and reapplied to the type-IV-collagen-SEPHAROSE until all material which will bind to the medium has been bound. The unbound fraction is collected and bound material is eluted from the type-IV-collagen-SEPHAROSE with 0.1 M HCl and discarded.

The material collected from the type-IV-collagen-SEPHAROSE is concentrated by ultrafiltration and dialyzed against a buffer such as 0.1 M BBS.

The above described steps for the purification for trapping antibody may be used in any order or in other combinations if desired.

Signaling Antibodies

Serum from immunized animals is collected, pooled and diluted with an equal volume of a buffer such as 0.1 M BBS. The diluted serum is applied to ECM-complex-SEPHAROSE equilibrated with a buffer such as 0.1 M BBS. The medium is then washed, in sequence, with a buffer such as 0.1 M glycine-HCl, pH 3.0, then 0.1 M glycine-HCl, pH 2.0. Antibodies which remain bound to the ECM-complex-SEPHAROSE are then eluted with 0.1 M HCl containing 0.1 M NaCl. The 0.1 M glycine-HCl, pH 3.0, 0.1 M glycine-HCl, pH 2.0 and 0.1 M HCl washes are collected, neutralized by the addition of 0.1 M BBS and pooled.

The neutralized antibody fraction is then applied to normal-serum-SEPHAROSE equilibrated with a buffer such as 0.1 M BBS. Unbound material is washed from the normal-serum-SEPHAROSE with a buffer such as 0.1 M BBS and reapplied to the normal-serum-SEPHAROSE until all material which will bind to the normal-serum-SEPHAROSE has been bound. The material which remains unbound is collected. Bound material is eluted from the normal-serum-SEPHAROSE with about 0.1 M HCl and discarded.

The unbound material from the normal-serum-SEPHAROSE is then applied to type-IV-collagen-SEPHAROSE equilibrated with a buffer such as 0.1 M BBS. Unbound material is washed from the type-IV-collagen-SEPHAROSE with a buffer such as 0.1 M BBS and reapplied to the type-IV-collagen-SEPHAROSE until all material which will bind to the medium has been bound. The unbound fraction is collected and bound material is eluted from the type-IV-collagen-SEPHAROSE with 0.1 M HCl and discarded.

The material collected is concentrated by ultrafiltration and dialyzed against a buffer such as 0.1 M BBS.

The above described steps for the purification for signaling antibody may be used in any order or in other combinations if desired.

Anti-Collagen Antibodies

Antiserum raised to each of the collagen subtypes are also collected, pooled within groups, and passed over affinity chromatograph medium corresponding to their respective collagens. The affinity purified antibodies are eluted from the affinity chromatograph medium by one of the methods described above and the eluate collected. The antibodies are then cross-adsorbed by passing them through affinity chromatograph medium coupled to the other collagens in order to remove cross reacting antibodies and to thereby form anti-type I, anti-type II, anti-type III, anti-type IV, anti-type V, and anti-type VI collagen antibodies. Each of the antibody preparations are specific for epitopes unique to the particular collagen subtype. The antibody preparations are concentrated and dialyzed against 0.1 M BBS.

These antibodies are particularly useful for analyzing collagen subtype distribution of the collagen fragments which make up a part of the ECM complex.

### Preparation of Monoclonal Antibodies

Mice, which are immunized by any of the above combination of methods, can be used to provide monoclonal antibodies. Other species of animal, which have also been successfully used for this purpose including rats, guinea pigs, and hamsters, are also suitable for use in the present invention.

Once animals have been immunized and determined to be hyper-immune they are boosted either by intravenous injection or by injection directly into the spleen. The animals are sacrificed 3 days after the booster injection. Their spleens and lymph nodes are harvested aseptically, and single cell suspensions are prepared by any of a variety of methods known to those skilled in the art, including teasing, forcing through wire mesh or repeated pipetting.

Antibody producing cells are immortalized by somatic cell fusion with any one of the currently available mouse myeloma cell lines which are HAT sensitive and non-antibody producing. Cells are fused by any of the procedures known to those skilled in the art, which include PEG fusion and electrofusion. Hybrid cells are selected for reactivity with the ECM complex and its constituent parts, cloned by limiting dilution, stabilized by long-term culture, subcloned, tested for antibody production by ELISA and cytoplasmic staining for IgG. The cells are further evaluated for antibodies, specificity, and the antibody isotope determined. All of these assays are performed using conventional ELISA methodology using commercially available reagents and supplies.

Clones which are promising on initial evaluation are expanded in mass stationary tissue culture and transferred either into the peritoneal cavity of Balb/c mice or into bioreactors for large scale production. The antibodies are purified from ascites fluids or concentrated bioreactor supernatants.

The monoclonal antibodies are then purified by collecting a precipitate which forms between 40 to 50% saturation (NH₄)₂SO₄. The precipitate is collected by centrifugation at about 10,000 to 20,000×g for 10 to 30 minutes. The antibodies are then purified by gel-filtration on a media such as SEPHACRYL (Pharmacia) or BIOGEL (BioRad Laboratories). The material which elutes in the void volume from the gel-filtration medium is then purified by ion exchange chromatography on hydroxyapatite (Calbiochem, La Jolla, Calif.) or ABx (Baker). Proteins are eluted from the ion exchange medium with a linear gradient from 0.001 to 0.2 M phosphate. IgG containing fractions are then purified on a DEAE cellulose medium such as DE-52 (Whatman) or DEAE SEPHADEX (Pharmacia). Proteins are eluted from the chromatography medium with a linear gradient from 0.05 to 0.3 M NaCl gradient. The IgG containing fractions are pooled and concentrated by ultrafiltration and tested for purity by SDS-PAGE. The pooled fractions are dialyzed into 0.1 M BBS.

When monoclonal antibodies are used, mixtures of monoclonal antibodies against different epitopes within the ECM complex are preferred, since the ECM complex isolated from different individuals may contain different constituents. Therefore, a mixture of monoclonal antibodies will result in an antibody preparation which is capable of recognizing ECM complex from different individuals.

### Immunoassay Assays

Immunoassays can be performed using either monoclonal antibodies, polyclonal antibodies, or a combination of the two and can use either intact antibodies, F(ab′)₂ fragments or F(ab′) fragments, or combinations thereof.

If F(ab′)₂ fragments are to be used, they are generated by dialyzing the antibodies from which they are to be prepared into 0.1 M acetate buffer or 0.1 M formic acid buffer at pH 3.5 to 4.5. Pepsin is added at a ratio of 2 to 3 mg per 100 mg of antibody and the mixture is incubated for 4 to 18 hours at a temperature from room temperature to 37° C. At the end of the incubation the mixture is neutralized and purified on a suitable gel-filtration media such as SEPHADEX, SEPHACRYL or BIOGEL.

The antibody preparation to be used as the “signalling” component in the immunoassay is conjugated to a suitable probe for use in either a fluorescent assay, an EIA (enzyme-immuno-assay) or a RIA (radio-immuno-assay). Probes suitable for use in the present invention include probes such as biotin, FITC (fluorescein isothiocynate), phycoerythrin, alkaline phosphatase, horseradish peroxidase and β-galactosidase. Biotinylated antibody are preferred in the present invention, and permit the use of an enzyme labelled avidin or streptavidin. Horseradish peroxidase conjugated to streptavidin is very commonly used for reaction with biotinylated antibody because it is cheap, stable, easy to use and generates strong signals with a variety of chromogenic substrates such as ABTS [2,2′ azinodi(ethylbenzthiazoline) sulfonate], TMB (tetramethyl benzidine) and OPD (o-phenylenediamine).

For the preparation of biotinylated antibodies, an antibody preparation is diluted to about 10 mg/ml and dialyzed into a high pH buffer (pH 9.5) such as 0.1 M carbonate or 0.1 M borate. Long chain NES biotin is purchased from any one of several commercial suppliers such as Sigma Chemical Co., and dissolved in DMF or other suitable solvent. The biotin solution is added to the antibody at a molar ratio of approximately 15:1, biotin:antibody. The mixture is incubated for about 30 to 60 minutes. At the end of the incubation free biotin is removed by dialysis or gel filtration of SEPHADEX G-25, BIOGEL P-10 or other suitable media. The biotin conjugated antibodies are stored at 4° C. with about 1% w/v bovine serum albumin (BSA) or other carrier protein added as a stabilizer.

Streptavidin, about 10 mg/ml, is dialyzed into a buffer such as 0.1 M carbonate buffer, pH 9, and horseradish peroxidase (HRP), about 10 mg/ml, is dialyzed into a buffer such as 0.1 M carbonate buffer, pH 8.0. Sodium periodate is added to the HRP to a final concentration of about 1 μg/ml. After about 2 to 4 hours at room temperature, the periodate is inactivated by the addition of 1 mg of dry G-25/ml streptavidin or antibody. After about 18 to 24 hours the conjugate is stabilized by the addition of 1 μg/ml sodium borohydride and purified by gel-filtration on a medium such as SUPEROSE 6 or SUPEROSE 12 (Pharmacia) to remove over- and under-conjugated material.

In another embodiment of the present invention HRP is conjugated to antibody, as described above.

The antibody preparations are evaluated for performance and test parameters are optimized simultaneously by analyzing the minimum and maximum detection limits and slope of a titration curve generated using purified ECM complex. The test is further optimized by comparing the values obtained for both normal reference serum and malignant reference serum. The assay can also use any of the polyclonal antibodies described above or a combination of polyclonal and monoclonal antibodies. The assay can be in the form of a competition immunoassay, a radioimmunoassay, immunofluorescence assay or any of the other assays well known to those skilled in the art.

A typical assay is performed as follows:

Ninety six well ELISA plates or 12 well ELISA strips are purchased commercially from any of several commercial suppliers, and the wells are passively coated with either various dilutions (between 2 to 10 μg/ml) for optimization, or with a predetermined optimal concentration, for testing, by incubating the wells with antibody solutions for about 18 to 24 hours at 4° C.

The wells are washed to remove excess, unbound antibody using any one of several commercially available manual or automatic washing systems using any appropriate washing buffer. Remaining binding sites on the plastic surface are blocked with a protein such as BSA.

Appropriate serial dilutions of serum samples and purified ECM complex are made and added to individual wells and incubated for about 2 to 24 hours at about 4 to 37° C.

The plates are again washed and various dilutions (from 1:2,000 to 1:20,000) or previously determined optimum dilutions of a 1 mg/ml biotin or enzyme labeled antibody preparation are added to the wells and incubated for about 2 to 24 hours at about 4 to 37° C.

The wells are again washed and evaluated for label remaining on the surface of the wells. When an enzyme label is used, any of the commercially available calorimetric or fluorescent substrates appropriate for the enzymatic activity of the specific enzyme chosen for conjugation is added and optical density or fluorescent intensity read at a wavelength appropriate for the particular substrate chosen. The optical density is read using one of many commercially available plate readers with automatic data reduction software capable of converting optical density units for test samples into concentration values by extrapolation onto a standard curve generated using purified ECM complex.

### Diagnosis of Malignant Disease

The above described assay can be used both to evaluate and optimize assay methods and to assay for the presence of ECM complex in test serum samples taken from normal donor patients suspected of having malignant disease, patients with malignant disease who are being monitored throughout the course of treatment or patients who are in remission. ECM complex concentrations detected by the immunoassay which are statistically significantly higher than baseline levels for a normal population are considered to be indicative of the presence of cancer in the patient from whom the test samples were taken.

### Diagnosis of Specific Cancers

The ECM complex released from different tissues are a result of proteolytic activity associated with tumor growth, and therefore, display a collagen subtype composition which reflects the tissue from which it is derived. In the present invention it has been determined that the collagen type distribution can also correlate with the site of metastases in addition to the site of the original tumor. The subtype profiles of the collagen component of ECM complex isolated from a normal subject and patients with various metastatic diseases are shown in Table I. The assay for analyzing the collagen type component of the ECM complex in patient serum samples is determined by collagen subtype specific antibodies to a 96 well ELISA plate, adding test samples to thereby bind ECM complex present in the test samples to the ELISA plate and completing the antibody/sandwich with biotinylated antibody specific for ECM complex as described above.

TABLE I

| Cancer Type/Antibody | I | II | III | IV | V |
|---|---|---|---|---|---|
| Normal | − | − | − | + | − |
| Breast Cancer | + | − | + | − | − |
| Ovarian Cancer | − | − | + | − | − |
| Ovarian Cancer | + | − | + | − | − |
| metastasized to bone | | | | | |
| Lung Cancer | + | − | − | − | − |
| Adenocarcinoma | − | − | + | − | + |

### EXAMPLE 1

### Purification of Crude ECM Complex

One liter of human ascites fluid from an ovarian cancer patient was collected, pooled and diluted to 4 liters with PBSE. The mixture was filtered through Whatman No. 1 paper (supplied by Whatman International, Ltd of Maidstone England) and clarified by centrifugation at 10,000×g for 30 minutes at 4° C. to remove cellular debris. The clarified solution was then equilibrated to 4° C. The equilibrated solution was brought to 1 M (NH₄)₂SO₄, by the addition of saturated (NH₄)₂SO₄ with constant stirring. The 1 M (NH₄)₂SO₄ solution was then stirred for 1 hour at 4° C. and allowed to stand overnight without stirring to allow the precipitate to settle. Most of the supernatant was removed by syphoning and discarded. The 1 M (NH₄)₂SO₄ precipitate was collected by centrifugation at 10,000×g for 10 minutes at 4° C. and then at 20,000×g for 30 minutes. The 1 M (NH₄)₂SO₄ precipitate was collected and resuspended in 100 ml PBSE, clarified by centrifugation at 20,000×g for 30 minutes at 4° C. and dialyzed against continuously running deionizing water for 24 hours. The precipitate which formed was collected by centrifugation at 20,000×g for 30 minutes at 4° C. The supernatant was discarded.

The precipitate was redissolved in 25 ml of 0.1 M BBS and clarified by centrifugation at 20,000×g for 30 minutes at 4° C. The supernatant was collected and analyzed by SDS-PAGE.

### EXAMPLE 2

### Purification of Crude ECM Complex

One liter of human pleural fluid from a lung cancer patient was collected, pooled and diluted to 4 liters with PBSE. The mixture was filtered through Whatman No. 1 paper and clarified by centrifugation at 10,000×g for 30 minutes at 4° C. to remove cellular debris. The clarified solution was then equilibrated to 4° C. The equilibrated solution was brought to 1.5 M (NH₄)₂SO₄, by the addition of saturated (NH₄)₂SO₄ with constant stirring. The 1.5 M (NH₄)₂SO₄ solution was then stirred for 1 hour at 4° C. and allowed to stand overnight without stirring to allow the precipitate to settle. Most of the supernatant was removed by syphoning and discarded. The 1.5 M (NH₄)₂SO₄ precipitate was collected by centrifugation at 10,000×g for 10 minutes at 4° C. and then at 20,000×g for 30 minutes. The 1.5 M (NH₄)₂SO₄ precipitate was collected and resuspended in 100 ml PBSE, clarified by centrifugation at 20,000×g for 30 minutes at 4° C. The precipitate was dissolved in 0.1 M BBS, 0.5 M NaCl and clarified by centrifugation at 20,000×g for 30 minutes at 4° C.

The supernatant was then brought to 2 M NaCl and mixed for 1 hour at 4° C. The precipitate which formed was collected by centrifugation at 20,000×g for 30 minutes at 4° C. The supernatant was then brought to 3 M NaCl and mixed for 1 hour at 4° C. The precipitate which formed was collected by centrifugation at 20,000×g for 30 minutes at 4° C. The supernatant was then brought to 4 M NaCl and mixed for 1 hour at 4° C. The precipitate which formed was collected by centrifugation at 20,000×g for 30 minutes at 4° C. The supernatant was then brought to 5 M NaCl and mixed for 1 hour at 4° C. The precipitate which formed was collected by centrifugation at 20,000×g for 30 minutes at 4° C.

The precipitates were each separately redissolved in 25 ml of PBSE and clarified by centrifugation at 20,000×g for 30 minutes at 4° C. The supernatants were collected and analyzed by SDS-PAGE.

### EXAMPLE 3

### Purification of Crude ECM Complex

One liter of human mastectomy drainage fluid (wound exudate) from a breast cancer patient was collected, pooled and diluted to 4 liters with PBSE. The mixture was filtered through Whatman No. 1 paper and clarified by centrifugation at 10,000×g for 30 minutes at 4° C. to remove cellular debris. The clarified solution was then brought to 4.5 to 5.0 M NaCl (a concentration approaching saturation). The mixture was stirred for 1 hour and allowed to stand overnight without stirring to allow the precipitate to settle. Most of the supernatant was removed by syphoning and discarded. The NaCl precipitated material was collected by centrifugation at 10,000×g for 10 minutes at 4° C. and then at 20,000×g for 30 minutes. The precipitate was collected and resuspended in 100 ml of 0.1 M BBS. The resuspended precipitate was brought to 1.2 M glycine and clarified by centrifugation at 20,000×g for 30 minutes at 4° C. The supernatant was then purified by gel filtration on SEPHAROSE 4B, equilibrated with 0.1 M BBS. The protein which eluted in the void volume was collected and purified by gel filtration on SEPHAROSE 6B, equilibrated with 0.1 M BBS. The protein which eluted in the void volume was collected and concentrated by ultra-filtration and analyzed by SDS-PAGE.

### EXAMPLE 4

### Purification of Crude ECM Complex

One liter of human thoracic fluid from a lung cancer patient was collected, pooled and diluted to 4 liters with PBSE. The mixture was filtered through Whatman No. 1 paper and clarified by centrifugation at 10,000×g for 30 minutes at 4° C. to remove cellular debris. The clarified solution was equilibrated to 4° C. The equilibrated solution was brought to 1.2 M (NH₄)₂SO₄, by the addition of saturated (NH₄)₂SO₄ with constant stirring. The 1.2 M (NH₄)₂SO₄ solution was then stirred for 1 hour at 4° C. and allowed to stand overnight without stirring to allow the precipitate to settle. Most of the supernatant was removed by syphoning and discarded. The 1.2 M (NH₄)₂SO₄ precipitate was collected by centrifugation at 10,000×g for 10 minutes at 4° C. and then at 20,000×g for 30 minutes. The 1.2 M (NH₄)₂SO₄ precipitate was collected and resuspended in 100 ml of 0.1 M BBS, clarified by centrifugation at 20,000×g for 30 minutes at 4° C. and dialyzed against continuously running deionizing water for 24 hours. Any precipitate which formed was collected by centrifugation at 20,000×g for 30 minutes at 4° C. and discarded. The supernatant was brought to 2 M glycine, by slowly adding dry glycine powder. The precipitate which formed was collected by centrifugation at 20,000×g for 30 minutes at 4° C., dissolved in 0.1 M BBS and analyzed by SDS-PAGE.

### EXAMPLE 5

### Purification of Normal Serum and Plasma

Normal human blood was collected either from volunteer donors or patients undergoing therapeutic phlebotomy for disorders unrelated to malignancy. Blood was either allowed to coagulate overnight at 4° C. and the serum collected, or was mixed with anticoagulants and the plasma collected. The serum or plasma was centrifuged at 1,000 to 2,000×g for 30 to 60 minutes and the supernatant was collected.

The resultant supernatant was conjugated to CNBr-activated SEPHAROSE.

### EXAMPLE 6

### Purification of Collagens

Human placentas (300 to 500 g) were collected from normal donors following delivery. The fresh placentas were minced, washed in saline, washed with water, washed with 0.5 M acetic acid and then with 0.5 M formic acid. The material remaining after the washing was mixed with 0.1 mg of pepsin for each gram of placenta in 0.5 M formic acid and incubated at 4° C. with constant mixing for 12 to 18 hours. At the end of the incubation the soluble material was centrifuged at 20,000×g for 30 minutes at 4° C. to remove undigested material. The supernatant was neutralized by the addition of sufficient 10 M NaOH, to inactivate the pepsin. The neutralized solution was precipitated by the addition of NaCl to a final concentration of 4.5 M. The resultant precipitate was collected by centrifugation at 20,000×g for 30 minutes at 4° C. The precipitate was resuspended in 0.5 M acetic acid and brought to 0.7 M NaCl.

The 1st precipitate and 1st supernatant were collected by centrifugation at 20,000×g for 10 minutes.

The 1st precipitate was dissolved in 1 M NaCl, clarified by centrifugation at 20,000×g for 10 minutes, and the supernatant was dialyzed against 0.02 M Tris-HCl, pH 7.5, 0.1% w/v (NH₄)₂SO₄ for 18 hours at 4° C. The solution was then dialyzed against 1 M NaCl for 18 hours at 4° C. and centrifuged at 20,000×g for 10 minutes to remove particulate matter. The supernatant was collected and dialyzed against 1.5 M NaCl for 18 hours at 4° C. The 2nd precipitate, which formed during the dialysis was collected by centrifugation 20,000×g for 10 minutes. The supernatant (2nd supernatant) was also collected.

The 2nd precipitate was dissolved in 1 M NaCl and the solution was clarified by centrifugation 20,000×g for 10 minutes. The resultant 3rd supernatant is collected by centrifugation at 20,000×g for 10 minutes and dialyzed against 1.5 M NaCl for 18 hours at 4° C. The 4th precipitate which formed was collected by centrifugation at 20,000×g for 10 minutes. The 4th precipitate included type III collagen.

Sodium chloride was added to the 2nd supernatant to a final concentration of 2 M. The precipitate which formed was collected by centrifugation at 20,000×g for 10 minutes and discarded. The 4th supernatant was brought to 4 M NaCl. The 5th precipitate which formed, and the resultant 5th supernatant were collected by centrifugation at 20,000×g for 10 minutes. The 5th supernatant included type II collagen and the 5th precipitate included type I collagen.

The 1st supernatant was dialyzed against 0.5 M acetic acid, 1.2 M NaCl for 18 hours at 4° C. and the 6th precipitate which formed and the resultant 6th supernatant were collected by centrifugation at 20,000×g for 10 minutes.

The 6th supernatant was dialyzed against 0.5 M acetic acid, 2 M NaCl for 18 hours at 4° C. The 7th precipitate which formed, which included type VI collagen, was collected by centrifugation at 20,000×g for 10 minutes. The supernatant was discarded.

The 7th precipitate was dissolved in 0.5 M acetic acid and dialyzed against 0.2 M sodium phosphate buffer, pH 9, for 18 hours at 4° C. The 8th precipitate which formed and the resultant 8th supernatant were collected by centrifugation at 20,000×g for 10 minutes. The 8th supernatant included type IV collagen. The 8th precipitate was dissolved in 1 M NaCl. The 9th precipitate which formed, which included type V collagen, was collected by centrifugation at 20,000×g for 10 minutes.

Each of the purified collagens were dialyzed against 0.5 M acetic acid for 18 hours at 4° C.

### EXAMPLE 7

### Preparation of CNBr-SEPHAROSE

SEPHAROSE 4B (Sigma Chemical Co.) was washed extensively with water, the slurry was collected and resuspended in an equal volume of 0.1 M carbonate, pH 11. Cyanogen bromide was dissolved in DMF and was added to the SEPHAROSE slurry to a final concentration of 33 mg/ml. The activation procedure was carried out on ice in a fume hood with constant monitoring of the pH of the solution. The pH was maintained at 11 by the addition of 10 M NaOH until the reaction stopped and the pH ceased dropping. The activated SEPHAROSE was washed in a vacuum funnel with chilled water and then chilled 0.1 M BBS.

### EXAMPLE 8

### Conjugation of Purified ECM Complex to SEPHAROSE

Activated SEPHAROSE, prepared as described in Example 7 was mixed with 10 mg of ECM complex prepared by the method described in Example 1 for each ml of packed volume of activated SEPHAROSE. The mixture was incubated at 4° C. overnight to allow coupling of the ECM complex to the activated SEPHAROSE to form ECM-complex-SEPHAROSE. Prior to use the ECM-complex-SEPHAROSE was blocked by washing with 1 M glycine for 2 hours at room temperature. The coupled ECM-complex-SEPHAROSE was washed with 0.1 M BBS and then packed into a column for use.

### EXAMPLE 9

### Conjugation of Normal Serum to SEPHAROSE

Activated SEPHAROSE, prepared as described in Example 7 was mixed with 10 mg of normal serum prepared by the method described in Example 5 for each ml of packed volume of activated SEPHAROSE. The mixture was incubated at 4° C. overnight to allow coupling of the normal serum to the activated SEPHAROSE to form normal-serum-SEPHAROSE. Prior to use the normal-serum-SEPHAROSE was blocked by washing with 1 M glycine for 2 hours at room temperature. The coupled normal-serum-SEPHAROSE was washed with 0.1 M BBS and then packed into a column for use.

### EXAMPLE 10

### Conjugation of Normal Plasma to SEPHAROSE

Activated SEPHAROSE, prepared as described in Example 7 was mixed with 10 mg of normal plasma prepared by the method described in Example 5 for each ml of packed volume of activated SEPHAROSE and incubated at 4° C. overnight to allow coupling of the normal plasma to the activated SEPHAROSE to form normal-plasma-SEPHAROSE. Prior to use the normal-plasma-SEPHAROSE was blocked by washing with 1 M glycine for 2 hours at room temperature. The coupled normal-plasma-SEPHAROSE was washed with 0.1 M BBS and then packed into a column for use.

### EXAMPLE 11

### Conjugation of Type I Collagen to SEPHAROSE

CNBr-activated SEPHAROSE, prepared by the method of Example 7, was added to 10 mg of type I collagen, prepared as described in Example 6, per ml of CNBr-activated SEPHAROSE, with gentle mixing. The mixture was incubated overnight at 4° C.

At the completion of the incubation the coupled type-I-collagen-SEPHAROSE was washed with 0.1 M BBS and remaining sites were blocked by incubating the coupled type-I-collagen-SEPHAROSE with 1 M glycine for 2 hours at room temperature. The coupled type-I-collagen-SEPHAROSE was washed with 0.1 M BBS and then packed into a column for use.

### EXAMPLE 12

### Conjugation of Type II Collagen to SEPHAROSE

CNBr-activated SEPHAROSE, prepared as described in Example 7 was added to 10 mg of type II collagen, prepared as described in Example 6, per ml of CNBr-activated SEPHAROSE, with gentle mixing. The mixture was incubated overnight at 4° C.

At the completion of the incubation the coupled type-II-collagen-SEPHAROSE was washed with 0.1 M BBS and remaining sites were blocked by incubating the coupled type-II-collagen-SEPHAROSE with 1 M glycine for 2 hours at room temperature. The coupled type-II-collagen-SEPHAROSE was washed with 0.1 M BBS and then packed into a column for use.

### EXAMPLE 13

### Conjugation of Type III Collagen to SEPHAROSE

CNBr-activated SEPHAROSE prepared as described in Example 7 was added to 10 mg of type III collagen, prepared as described in Example 6, per ml of CNBr-activated SEPHAROSE, with gentle mixing. The mixture was incubated overnight at 4° C.

At the completion of the incubation the coupled type-III-collagen-SEPHAROSE was washed with 0.1 M BBS and remaining sites were blocked by incubating the coupled type-III-collagen-SEPHAROSE with 1 M glycine for 2 hours at room temperature. The coupled type-III-collagen-SEPHAROSE was washed with 0.1 M BBS and then packed into a column for use.

### EXAMPLE 14

### Conjugation of Type IV Collagen to SEPHAROSE

CNBr-activated SEPHAROSE, prepared as described in Example 7, was added to 10 mg of type IV collagen, prepared as described in Example 6, per ml of CNBr-activated SEPHAROSE, with gentle mixing. The mixture was incubated overnight at 4° C.

At the completion of the incubation the coupled type-IV-collagen-SEPHAROSE was washed with 0.1 M BBS and remaining sites were blocked by incubating the coupled type-IV-collagen-SEPHAROSE with 1 M glycine for 2 hours at room temperature. The coupled type-IV-collagen-SEPHAROSE was washed with 0.1 M BBS and then packed into a column for use.

### EXAMPLE 15

### Conjugation of Type V Collagen to SEPHAROSE

CNBr-activated SEPHAROSE, prepared as described in Example 7, was added to 10 mg of type V collagen, prepared as described in Example 6, per ml of CNBr-activated SEPHAROSE, with gentle mixing. The mixture was incubated overnight at 4° C.

At the completion of the incubation the coupled type-V-collagen-SEPHAROSE was washed with 0.1 M BBS and remaining sites were blocked by incubating the coupled type-V-collagen-SEPHAROSE with 1 M glycine for 2 hours at room temperature. The coupled type-V-collagen-SEPHAROSE was washed with 0.1 M BBS and then packed into a column for use.

### EXAMPLE 16

### Conjugation of Type VI Collagen to SEPHAROSE

CNBr-activated SEPHAROSE, prepared as described in Example 7, 10 mg of type VI collagen, prepared as described in Example 6, per ml of CNBr-activated SEPHAROSE, with gentle mixing. The mixture was incubated overnight at 4° C.

At the completion of the incubation the coupled type-VI-collagen-SEPHAROSE was washed with 0.1 M BBS and remaining sites were blocked by incubating the coupled type-VI-collagen-SEPHAROSE with 1 M glycine for 2 hours at room temperature. The coupled type-VI-collagen-SEPHAROSE was washed with 0.1 M BBS and then packed into a column for use.

### EXAMPLE 17

### Preparation of Polyclonal Antibodies Against Purified ECM Complex in Rabbits

Eighteen New Zealand white rabbits were injected with pooled, purified antigen, in equal parts, purified by the procedure of Examples 1-4. For the primary immunization 1 to 2 mg of antigen was mixed with 0.5 ml Freund's Incomplete Adjuvant and mixed by sonication. Injections were given subcutaneously at multiple injection sites. After a six week interval a second injection of 0.25 to 0.5 mg of antigen in Freund's Incomplete Adjuvant was given subcutaneously at multiple injection sites. After six weeks the animals were bled via the proximal ear vein and 25 to 50 ml of blood was collected for each bleed. This injection/bleed procedure was continued for 6 to 12 months.

The blood, immediately after collection, was coagulated at 4° C. and the serum collected and centrifuged at 2,000×g for 30 minutes at 4° C. The clarified supernatant was collected and 0.1% w/v sodium azide was added.

### EXAMPLE 18

### Preparation of Polyclonal Antibodies Against Purified Collagen Subtypes in Rabbits

Eighteen New Zealand white rabbits were divided into 6 groups and each group was immunized as described in Example 17 however, collagen types I, II, III, IV, V or VI prepared as described in Example 6 were used in place of the ECM complex.

The blood was separately collected and coagulated at 4° C. and the serum collected and centrifuged at 2,000×g for 30 minutes at 4° C. The clarified supernatant was collected and 0.1% w/v sodium azide was added.

### EXAMPLE 19

### Preparation of Polyclonal Antibodies Against ECM Complex in Rabbits

Twelve New Zealand white rabbits were divided into 4 groups and each group was immunized as described in Example 17 except ECM complex from pleural fluid, ascites fluid or lung fluid from patients were used.

The blood was separately collected and coagulated at 4° C. and the serum collected and centrifuged at 2,000×g for 30 minutes at 4° C. The clarified supernatant was collected and 0.1% w/v sodium azide was added.

### EXAMPLE 20

### Purification of Polyclonal Antibodies-Trapping Antibodies

One liter of pooled serum prepared as described in Example 17 was diluted with 1 liter of 0.1 M BBS, pH 8.2. The diluted serum was applied to ECM-complex-SEPHAROSE prepared as described in Example 8, equilibrated with 0.1 M BBS. Unbound material was washed from the ECM-complex-SEPHAROSE with 0.1 M BBS and the eluate was reapplied to the ECM-complex-SEPHAROSE. This process was repeated twice. The unbound fraction was then collected. Bound material was eluted from the ECM-complex-SEPHAROSE with 0.1 M glycine-HCl, pH 3.0, then 0.1 M glycine-HCl, pH 2.0 and then 0.1 M HCl containing 0.1 M NaCl. The 0.1 M glycine-HCl, pH 3.0, 0.1 M glycine-HCl, pH 2.0 and 0.1 M HCl washes were collected, neutralized by the addition of 0.1 M BBS and pooled.

The neutralized antibody was then applied to type-IV-collagen-SEPHAROSE prepared as described in Example 14, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the type-IV-collagen-SEPHAROSE was then applied to normal-serum-SEPHAROSE prepared as described in Example 9, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the normal-serum-SEPHAROSE was collected, concentrated by ultra-filtration, dialyzed against 0.1 M BBS and stored at 4° C. until required.

### EXAMPLE 21

### Purification of Polyclonal Antibodies-Trapping Antibodies

One liter of pooled serum prepared as described in Example 17 was diluted with 1 liter of 0.1 M BBS, pH 8.2. The diluted serum was applied to ECM-complex-SEPHAROSE prepared as described in Example 8, equilibrated with 0.1 M BBS. Unbound material was washed from the ECM-complex-SEPHAROSE with 0.1 M BBS and the eluate was reapplied to the ECM-complex-SEPHAROSE. This process was repeated twice. The unbound fraction was then collected. Bound material was eluted from the ECM-complex-SEPHAROSE with 0.1 M glycine-HCl, pH 3.0, then 0.1 M glycine-HCl, pH 2.0 and then 0.1 M HCl containing 0.1 M NaCl. The 0.1 M glycine-HCl, pH 3.0, 0.1 M glycine-HCl, pH 2.0 and 0.1 M HCl washes were collected, neutralized by the addition of 0.1 M BBS and pooled.

The neutralized antibody was then applied to type-IV-collagen-SEPHAROSE prepared as described in Example 14, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the type-IV-collagen-SEPHAROSE was then applied to normal-serum-SEPHAROSE prepared as described in Example 9, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the normal-serum-SEPHAROSE was then applied to normal-plasma-SEPHAROSE prepared as described in Example 10, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the normal-plasma-SEPHAROSE was collected, concentrated by ultra-filtration, dialyzed against 0.1 M BBS and stored at 4° C. until required.

### EXAMPLE 22

### Purification of Polyclonal Antibodies-Trapping Antibodies

One liter of pooled serum prepared as described in Example 17 was diluted with 1 liter of 0.1 M BBS, pH 8.2. The diluted serum was applied to type-VI-collagen-SEPHAROSE prepared as described in Example 16, equilibrated with 0.1 M BBS. Unbound material was washed from the type-VI-collagen-SEPHAROSE with 0.1 M BBS and the eluate was reapplied to the type-VI-collagen-SEPHAROSE. This process was repeated twice. The unbound fraction was then collected. Bound material was eluted from the type-VI-collagen-SEPHAROSE with 0.1 M glycine-HCl, pH 3.0, then 0.1 M glycine-HCl, pH 2.0 and then 0.1 M HCl containing 0.1 M NaCl. The 0.1 M glycine-HCl, pH 3.0, 0.1 M glycine-HCl, pH 2.0 and 0.1 M HCl washes were collected, neutralized by the addition of 0.1 M BBS and pooled.

The neutralized antibody was then applied to type-IV-collagen-SEPHAROSE prepared as described in Example 14, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the type-IV-collagen-SEPHAROSE was then applied to normal-serum-SEPHAROSE prepared as described in Example 9, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the normal-serum-SEPHAROSE was collected, concentrated by ultra-filtration, dialyzed against 0.1 M BBS and stored at 4° C. until required.

### EXAMPLE 23

### Purification of Polyclonal Antibodies-Signaling Antibodies

One liter of pooled serum prepared as described in Example 17 was diluted with 1 liter of 0.1 M BBS, pH 8.2. The diluted serum was applied to ECM-complex-SEPHAROSE prepared as described in Example 8, equilibrated with 0.1 M BBS. Unbound material was washed from the ECM-complex-SEPHAROSE with 0.1 M BBS and the eluate was reapplied to the ECM-complex-SEPHAROSE. This process was repeated twice. The unbound fraction was then collected. Bound material was eluted from the ECM-complex-SEPHAROSE with 0.1 M glycine, pH 3.0, 0.1 M glycine-HCl, pH 2.0 and then 0.1 M HCl containing 0.1 M NaCl. The 0.1 M glycine-HCl, pH 3.0, 0.1 M glycine-HCl, pH 2.0 and 0.1 M HCl washes are collected, neutralized by the addition of 0.1 M BBS and pooled.

The neutralized antibody was then applied to type-IV-collagen-SEPHAROSE prepared as described in Example 14, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the type-IV-collagen-SEPHAROSE was then applied to normal-serum-SEPHAROSE prepared as described in Example 9, equilibrated with 0.1 M BBS. Unbound material was washed from the medium with 0.1 M BBS and the eluate was reapplied to the medium. This process was repeated twice. The unbound fraction was then collected. Bound material was eluted from the medium with 0.1 M HCl and discarded.

The unbound fraction from the normal-serum-SEPHAROSE was collected, concentrated by ultra-filtration, dialyzed against 0.1 M BBS and stored at 4° C. until required.

### EXAMPLE 24

### Purification of Polyclonal Antibodies Against Collagen Type I

One liter of pooled serum prepared as described in Example 18 was diluted with 1 liter of 0.1 M BBS, pH 8.2. The diluted serum was applied to type-I-collagen-SEPHAROSE prepared as described in Example 11, equilibrated with 0.1 M BBS. Unbound material was washed from the type-I-collagen-SEPHAROSE with 0.1 M BBS and the eluate was reapplied to the type-I-collagen-SEPHAROSE. This process was repeated twice. The unbound fraction was discarded. Bound material was eluted from the type-I-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-II-collagen-SEPHAROSE prepared as described in Example 12, equilibrated with 0.1 M BBS. Unbound material was washed from the medium with 0.1 M BBS and the eluate was reapplied to the medium. The unbound fraction was then collected. Bound material was eluted from the type-II-collagen-SEPHAROSE with 0.1 M HCl and discarded.

The unbound fraction from the type-II-collagen-SEPHAROSE was then applied to type-III-collagen-SEPHAROSE prepared as described in Example 13, equilibrated with 0.1 M BBS. Unbound material was washed from the medium with 0.1 M BBS and the eluate was reapplied to the medium. The unbound fraction was then collected. Bound material was eluted from the type-III-collagen-SEPHAROSE with 0.1 M HCl and discarded.

The unbound fraction from the type-III-collagen-SEPHAROSE was then applied to type-IV-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-IV-collagen-SEPHAROSE was then applied to type-V-collagen-SEPHAROSE prepared as described in Example 15, equilibrated with 0.1 M BBS. Unbound material was washed from the medium with 0.1 M BBS and the eluate was reapplied to the medium. The unbound fraction was then collected. Bound material was eluted from the type-V-collagen-SEPHAROSE with 0.1 M HCl and discarded.

The unbound fraction from the type-V-collagen-SEPHAROSE was then applied to type-VI-collagen-SEPHAROSE prepared as described in Example 16, equilibrated with 0.1 M BBS. Unbound material was washed from the medium with 0.1 M BBS and the eluate was reapplied to the medium. The unbound fraction was then collected. Bound material was eluted from the type-VI-collagen-SEPHAROSE with 0.1 M HCl and discarded.

The unbound fraction from the type-VI-collagen-SEPHAROSE was collected, concentrated by ultra-filtration, dialyzed against 0.1 M BBS and stored at 4° C. until required.

### EXAMPLE 25

### Purification of Polyclonal Antibodies Against Collagen Type II

1 liter of pooled serum prepared as described in Example 18 was diluted with 1 liter of 0.1 M BBS, pH 8.2. The diluted serum was applied to type-II-collagen-SEPHAROSE prepared as described in Example 12, equilibrated with 0.1 M BBS. Unbound material was washed from the type-II-collagen-SEPHAROSE with 0.1 M BBS and the eluate was reapplied to the type-II-collagen-SEPHAROSE. This process was repeated twice. The unbound fraction was discarded. Bound material was eluted from the type-II-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-I-collagen-SEPHAROSE prepared as described in Example 11, equilibrated with 0.1 M BBS. Unbound material was washed from the medium with 0.1 M BBS and the eluate was reapplied to the medium. The unbound fraction was then collected. Bound material was eluted from the type-I-collagen-SEPHAROSE with 0.1 M HCl and discarded.

The unbound fraction from the type-I-collagen-SEPHAROSE was then applied to type-III-collagen-SEPHAROSE prepared as described in Example 7, equilibrated with 0.1 M BBS. Unbound material was washed from the medium with 0.1 M BBS and the eluate was reapplied to the medium. The unbound fraction was then collected. Bound material was eluted from the type-III-collagen-SEPHAROSE with 0.1 M HCl and discarded.

The unbound fraction from the type-III-collagen-SEPHAROSE was then applied to type-IV-collagen-SEPHAROSE prepared as described in Example 13, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-IV-collagen-SEPHAROSE was then applied to type-IV-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-IV-collagen-SEPHAROSE was then applied to type-V-collagen-SEPHAROSE prepared as described in Example 15, equilibrated with 0.1 M BBS. Unbound material was washed from the medium with 0.1 M BBS and the eluate was reapplied to the medium. The unbound fraction was then collected. Bound material was eluted from the type-V-collagen-SEPHAROSE with 0.1 M HCl and discarded.

The unbound fraction from the type-V-collagen-SEPHAROSE was then applied to type-VI-collagen-SEPHAROSE prepared as described in Example 16, equilibrated with 0.1 M BBS. Unbound material was washed from the medium with 0.1 M BBS and the eluate was reapplied to the medium. The unbound fraction was then collected. Bound material was eluted from the type-VI-collagen-SEPHAROSE with 0.1 M HCl and discarded.

The unbound fraction from the type-VI-collagen-SEPHAROSE was collected, concentrated by ultra-filtration, dialyzed against 0.1 M BBS and stored at 4° C. until required.

### EXAMPLE 26

### Purification of Polyclonal Antibodies Against Collagen Type III

One liter of pooled serum prepared as described in Example 18 was diluted with 1 liter of 0.1 M BBS, pH 8.2. The diluted serum was applied to type-III-collagen-SEPHAROSE prepared as described in Example 13, equilibrated with 0.1 M BBS. Unbound material was washed from the type-III-collagen-SEPHAROSE with 0.1 M BBS and the eluate was reapplied to the type-III-collagen-SEPHAROSE. This process was repeated twice. The unbound fraction was discarded. Bound material was eluted from the type-III-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-I-collagen-SEPHAROSE prepared as described in Example 11, equilibrated with 0.1 M BBS. Unbound material was washed from the medium with 0.1 M BBS and the eluate was reapplied to the medium. The unbound fraction was then collected. Bound material was eluted from the type-I-collagen-SEPHAROSE with 0.1 M HCl and discarded.

The unbound fraction from the type-I-collagen-SEPHAROSE was then applied to type-II-collagen-SEPHAROSE prepared as described in Example 12, equilibrated with 0.1 M BBS. Unbound material was washed from the medium with 0.1 M BBS and the eluate was reapplied to the medium. The unbound fraction was then collected. Bound material was eluted from the type-II-collagen-SEPHAROSE with 0.1 M HCl and discarded.

The unbound fraction from the type-II-collagen-SEPHAROSE was then applied to type-IV-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-IV-collagen-SEPHAROSE was then applied to type-V-collagen-SEPHAROSE prepared as described in Example 15, equilibrated with 0.1 M BBS. Unbound material was washed from the medium with 0.1 M BBS and the eluate was reapplied to the medium. The unbound fraction was then collected. Bound material was eluted from the type-V-collagen-SEPHAROSE with 0.1 M HCl and discarded.

The unbound fraction from the type-V-collagen-SEPHAROSE was then applied to type-VI-collagen-SEPHAROSE prepared as described in Example 16, equilibrated with 0.1 M BBS. Unbound material was washed from the medium with 0.1 M BBS and the eluate was reapplied to the medium. The unbound fraction was then collected. Bound material was eluted from the type-VI-collagen-SEPHAROSE with 0.1 M HCl and discarded.

The unbound fraction from the type-VI-collagen-SEPHAROSE was collected, concentrated by ultra-filtration, dialyzed against 0.1 M BBS and stored at 4° C. until required.

### EXAMPLE 27

### Purification of Polyclonal Antibodies Against Collagen Type IV

One liter of pooled serum prepared as described in Example 18 was diluted with 1 liter of 0.1 M BBS, pH 8.2. The diluted serum was applied to type-IV-collagen-SEPHAROSE prepared as described in Example 14, equilibrated with 0.1 M BBS. Unbound material was washed from the type-IV-collagen-SEPHAROSE with 0.1 M BBS and the eluate was reapplied to the type-IV-collagen-SEPHAROSE. This process was repeated twice. The unbound fraction was discarded. Bound material was eluted from the type-IV-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-I-collagen-SEPHAROSE prepared as described in Example 11, equilibrated with 0.1 M BBS. Unbound material was washed from the medium with 0.1 M BBS and the eluate was reapplied to the medium. The unbound fraction was then collected. Bound material was eluted from the type-I-collagen-SEPHAROSE with 0.1 M HCl and discarded.

The unbound fraction from the type-I-collagen-SEPHAROSE was then applied to type-II-collagen-SEPHAROSE prepared as described in Example 12, equilibrated with 0.1 M BBS. Unbound material was washed from the medium with 0.1 M BBS and the eluate was reapplied to the medium. The unbound fraction was then collected. Bound material was eluted from the type-II-collagen-SEPHAROSE with 0.1 M HCl and discarded.

The unbound fraction from the type-II-collagen-SEPHAROSE was then applied to type-III-collagen-SEPHAROSE prepared as described in Example 13, equilibrated with 0.1 M BBS. Unbound material was washed from the medium with 0.1 M BBS and the eluate was reapplied to the medium. The unbound fraction was then collected. Bound material was eluted from the type-III-collagen-SEPHAROSE with 0.1 M HCl and discarded.

The unbound fraction from the type-III-collagen-SEPHAROSE was then applied to type-V-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-V-collagen-SEPHAROSE with 0.1 M HCl and discarded.

The unbound fraction from the type-V-collagen-SEPHAROSE was then applied to type-VI-collagen-SEPHAROSE prepared as described in Example 16, equilibrated with 0.1 M BBS. Unbound material was washed from the medium with 0.1 M BBS and the eluate was reapplied to the medium. The unbound fraction was then collected. Bound material was eluted from the type-VI-collagen-SEPHAROSE with 0.1 M HCl and discarded.

The unbound fraction from the type-VI-collagen-SEPHAROSE was collected, concentrated by ultra-filtration, dialyzed against 0.1 M BBS and stored at 4° C. until required.

### EXAMPLE 28

### Purification of Polyclonal Antibodies Against Collagen Type V

One liter of pooled serum prepared as described in Example 18 was diluted with 1 liter of 0.1 M BBS, pH 8.2. The diluted serum was applied to type-V-collagen-SEPHAROSE prepared as described in Example 15, equilibrated with 0.1 M BBS. Unbound material was washed from the type-V-collagen-SEPHAROSE with 0.1 M BBS and the eluate was reapplied to the type-V-collagen-SEPHAROSE. This process was repeated twice. The unbound fraction was discarded. Bound material was eluted from the type-V-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-I-collagen-SEPHAROSE prepared as described in Example 11, equilibrated with 0.1 M BBS. Unbound material was washed from the medium with 0.1 M BBS and the eluate was reapplied to the medium. The unbound fraction was then collected. Bound material was eluted from the type-I-collagen-SEPHAROSE with 0.1 M HCl and discarded.

The unbound fraction from the type-I-collagen-SEPHAROSE was then applied to type-II-collagen-SEPHAROSE prepared as described in Example 12, equilibrated with 0.1 M BBS. Unbound material was washed from the medium with 0.1 M BBS and the eluate was reapplied to the medium. The unbound fraction was then collected. Bound material was eluted from the type-II-collagen-SEPHAROSE with 0.1 M HCl and discarded.

The unbound fraction from the type-II-collagen-SEPHAROSE was then applied to type-III-collagen-SEPHAROSE prepared as described in Example 13, equilibrated with 0.1 M BBS. Unbound material was washed from the medium with 0.1 M BBS and the eluate was reapplied to the medium. The unbound fraction was then collected. Bound material was eluted from the type-III-collagen-SEPHAROSE with 0.1 M HCl and discarded.

The unbound fraction from the type-III-collagen-SEPHAROSE was then applied to type-IV-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-III-collagen-SEPHAROSE was then applied to type-VI-collagen-SEPHAROSE prepared as described in Example 16, equilibrated with 0.1 M BBS. Unbound material was washed from the medium with 0.1 M BBS and the eluate was reapplied to the medium. The unbound fraction was then collected. Bound material was eluted from the type-VI-collagen-SEPHAROSE with 0.1 M HCl and discarded.

The unbound fraction from the type-VI-collagen-SEPHAROSE was collected, concentrated by ultra-filtration, dialyzed against 0.1 M BBS and stored at 4° C. until required.

### EXAMPLE 29

### Purification of Polyclonal Antibodies Against Collagen Type VI

One liter of pooled serum prepared as described in Example 18 was diluted with 1 liter of 0.1 M BBS, pH 8.2. The diluted serum was applied to type-VI-collagen-SEPHAROSE prepared as described in Example 16, equilibrated with 0.1 M BBS. Unbound material was washed from the type-VI-collagen-SEPHAROSE with 0.1 M BBS and the eluate was reapplied to the type-VI-collagen-SEPHAROSE. This process was repeated twice. The unbound fraction was discarded. Bound material was eluted from the type-VI-collagen-SEPHAROSE with 0.1 M HCl, containing 0.1 M NaCl. The antibody which eluted with 0.1 M HCl, containing 0.1 M NaCl was collected and neutralized by the addition of 0.1 M BBS.

The neutralized antibody was then applied to type-I-collagen-SEPHAROSE prepared as described in Example 11, equilibrated with 0.1 M BBS. Unbound material was washed from the medium with 0.1 M BBS and the eluate was reapplied to the medium. The unbound fraction was then collected. Bound material was eluted from the type-I-collagen-SEPHAROSE with 0.1 M HCl and discarded.

The unbound fraction from the type-I-collagen-SEPHAROSE was then applied to type-II-collagen-SEPHAROSE prepared as described in Example 12, equilibrated with 0.1 M BBS. Unbound material was washed from the medium with 0.1 M BBS and the eluate was reapplied to the medium. The unbound fraction was then collected. Bound material was eluted from the type-II-collagen-SEPHAROSE with 0.1 M HCl and discarded.

The unbound fraction from the type-II-collagen-SEPHAROSE was then applied to type-III-collagen-SEPHAROSE prepared as described in Example 13, equilibrated with 0.1 M BBS. Unbound material was washed from the medium with 0.1 M BBS and the eluate was reapplied to the medium. The unbound fraction was then collected. Bound material was eluted from the type-III-collagen-SEPHAROSE with 0.1 M HCl and discarded.

The unbound fraction from the type-III-collagen-SEPHAROSE was then applied to type-IV-collagen-SEPHAROSE prepared as described in Example 14, equilibrated with 0.1 M BBS. Unbound material was washed from the medium with 0.1 M BBS and the eluate was reapplied to the medium. The unbound fraction was then collected. Bound material was eluted from the type-IV-collagen-SEPHAROSE with 0.1 M HCl and discarded.

The unbound fraction from the type-IV-collagen-SEPHAROSE was then applied to type-V-collagen-SEPHAROSE prepared as described in Example 15, equilibrated with 0.1 M BBS. Unbound material was washed from the medium with 0.1 M BBS and the eluate was reapplied to the medium. The unbound fraction was then collected. Bound material was eluted from the type-V-collagen-SEPHAROSE with 0.1 M HCl and discarded.

The unbound fraction from the type-V-collagen-SEPHAROSE was collected, concentrated by ultra-filtration, dialyzed against 0.1 M BBS and stored at 4° C. until required.

### EXAMPLE 30

### Conjugation of Antibodies to Biotin

Anti-ECM antibodies, prepared in accordance with Example 23, were diluted to 10 mg/ml with 0.1 M carbonate buffer, pH 9.5 and dialyzed against 0.1 M carbonate buffer, pH 9.5, overnight at 4° C. Biotinamidocaproate N-hydroxy succinimide ester (Sigma Chemical Co., Cat No B2643) was dissolved in N,N-dimethylformamide (Sigma Chemical Co., Cat No. D4254) and added to the antibody solution at a molar ratio of 15:1. The mixture was incubated at room temperature for 60 minutes. At the end of the incubation the mixture was applied to Sephadex G-25 (Pharmacia) packed into a PD-10 column (Pharmacia) and equilibrated with 0.1 M BBS. Material in the void volume was collected and 1% w/v BSA was added and the conjugate was sterilized by filtration and stored at 4° C.

### EXAMPLE 31

### Preparation of Streptavidin-Horseradish Peroxidase Conjugate

Ten mg/ml Streptavidin (Boehinger Mannheim Chemical Co., 152-0679) was dialyzed into 0.1 M carbonate buffer, pH 9.0 and 10 mg/ml horseradish peroxidase (HRP; Sigma Chemical Co., Cat. No. P8415) was dialyzed into 0.1 M carbonate buffer, pH 8.0 overnight at 4° C. Sodium periodate (Sigma Chemical Co., Cat. No. S1878) was added to the HRP solution to about 1 μg/ml and incubated at room temperature for about 2-4 hours. One g of dry SEPHADEX G-25 (Sigma Chemical Co., Cat No. G25-150) was added per ml of reaction mixture to inactivate the periodate and the streptavidin was added to the mixture. After 18-24 hours at 4° C., 1 μg/ml sodium borohydride (Sigma Chemical Co., Cat. No. S9125) was added and the conjugate was purified by gel-filtration on a matrix of SUPEROSE 6, and then on SUPEROSE 12 to exclude over-conjugated and under-conjugated streptavidin.

### EXAMPLE 32

### Studies Using A Cancer Pan-Marker

Ninety-six well immunoassay plates (supplied by Labsystems Inc. Marlboro MA: HIGH BIND COMBIPLATE, Cat. No. 950-29000P) were coated with purified anti-collagen type VI antibodies by incubating each well to be coated with 0.1 ml of a 10 μg/ml antibody solution in 0.1 M BBS, pH 8.8, overnight at 4° C.

At the end of the incubation the unbound antibody was washed from the wells with 0.02% v/v TWEEN 20 (Sigma Chemical Co., Cat. No. P1379), 0.01% v/v TRITON X100 (Sigma Chemical Co., Cat. No. X-100) and 0.1 M BBS, pH 8.3 or with STABILCOAT (Bio-Metric Systems, Inc. Eden Prairie, MN Cat No. 01-1000). The wells were then washed with 0.05% w/v TWEEN 20 in 0.1 M BBS to remove unbound BSA. Samples to be tested were diluted 1:500 with 0.02% v/v TWEEN 20, 0.01% v/v TRITON X100, 1.0% w/v BSA and 0.01% w/v THIMEROSAL (Sigma Chemical Co., Cat. No. T5125) in 0.1 M BBS, pH 8.3, and 0.1 ml was added to duplicate wells of the coated plates. The plates were then incubated at 37° C. for 2 hours. At the end of the incubation the wells were washed with 0.02% w/v TWEEN 20, 0.01% v/v TRITON X100 in 0.1 M BBS. Then 0.1 ml of biotinylated-anti-ECM antibodies, diluted 1:5,000 from a 1 mg/ml stock, was added to each well and the plates were incubated for 1 hour at 37° C. At the end of the incubation the wells were washed as described above and 0.1 ml of horseradish peroxidase (HRP)-streptavidin diluted 1:10,000 was added to each well. The plates were incubated at 37° C. for 1 hour. Unbound HRP-streptavidin was removed by washing the wells as described above and then 0.1 ml of 3,3′, 5,5′-tetramethylbenzidine (TMB; KPL, Gaithersburg, Md. Cat. No. 50-76-05) was added to each well.

The rate of color development at 650 nm was read on an automatic plate reader (Molecular Devices Vmax Plate Reader, Menlo Park, Calif.) at room temperature over an interval of 5 minutes. The raw data were reported as mOD/minute. Standard stock solutions prepared as 0, 4, 8, 16, 32, 64, 128 and 256 μg/ml ECM complex in diluent buffer, described above, were also diluted 1:500 and assayed in the same plates as the unknown samples. When completed, the raw data for the standards were displayed in a four parameter mode. The raw data from the unknown serum samples were interpolated from the standard curve and reported as ECM complex concentrations in ng/ml.

A positive result was one which was greater than 13 ng/ml, and indicated the presence of the ECM complex in the body fluid of the subject, and therefore, the presence of cancer.

### EXAMPLE 33

### Studies Using A Cancer Pan-Marker

Trapping antibodies which were purified according to the procedure set out in Example 21 and biotinylated signalling antibodies which were purified according to the procedure set out in Examples 23 and 30, along with streptavidin-horseradish peroxidase prepared as described in Example 31 were used to perform an assay according to the procedure set out in Example 32. The results obtained are presented in Table II.

TABLE II

| Class | No. Neg./Total | % Negative |
|---|---|---|
| Normal serum | 153/154 | 99.4 |
| Remission | | |
| Lung Cancer | 5/5 | 100 |
| Breast Cancer | 7/8 | 87.5 |
| Prostate Cancer | 9/14 | 64.3 |
| Colon Cancer | 1/1 | — |
| Adrenal Cell Cancer | 1/1 | — |
| Thrombocytosis | 2/2 | — |
| Bronchial Cancer | 1/1 | — |
| Melanoma | 1/1 | — |
| Ovarian Cancer | 1/1 | — |

### EXAMPLE 34

### Studies Using A Cancer Pan-Marker

Trapping antibodies which were purified according to the procedure set out in Example 21 and biotinylated signalling antibodies which were purified according to the procedure set out in Examples 23 and 30, along with streptavidin-horseradish peroxidase prepared as described in Example 31 were used to perform an assay according to the procedure set out in Example 32. The results obtained are presented in Table III.

TABLE III

| Tumor type | No. Pos./Total | % Sensitivity |
|---|---|---|
| Lung Cancer | 43/45 | 95.6 |
| Breast Cancer | 18/19 | 94.7 |
| Prostate Cancer | 5/9 | 55.6 |
| Colon Cancer | 4/4 | 100 |
| Esophageal Cancer | 1/1 | — |
| Mesothelioma | 1/1 | — |
| Lymphoma | 3/3 | 100 |
| Leukemia | 3/4 | 75 |
| Multiple Melanoma | 1/1 | — |
| Adeno Cancer (unknown | 1/1 | — |
| source) | | |
| Ovarian Cancer | 3/3 | 100 |

### EXAMPLE 35

### Studies Using A Cancer Pan-Marker

Trapping antibodies which were purified according to the procedure set out in Example 22 and biotinylated signalling antibodies which were purified according to the procedure set out in Examples 23 and 30, along with the streptavidin-horseradish peroxidase prepared as described in Example 31 were used to perform an assay according to the procedure set out in Example 32. The results obtained are presented in Table IV.

TABLE IV
ECM Serum Concentrations (ng/ml)

| | Lung | | Lung | | Lung |
|---|---|---|---|---|---|
| Normal | Cancer | Normal | Cancer | Normal | Cancer |
| 9 | 180 | 7 | 37 | 8 | |
| 13 | 38 | 8 | 105 | 11 | |
| 9 | 21 | 5 | 30 | 8 | |
| 6 | 41 | 5 | 115 | 11 | |
| 5 | 48 | 7 | 27 | 9 | |
| 10 | 129 | 7 | 56 | 10 | |
| 8 | 54 | 6 | 35 | 8 | |
| 11 | 87 | 10 | 53 | 11 | |
| 8 | 69 | 11 | 11 | 11 | |
| 10 | 43 | 14 | 102 | 10 | |
| 7 | 106 | 8 | 164 | 12 | |
| 10 | 121 | 14 | 16 | 9 | |
| 9 | 71 | 8 | 17 | | |
| 7 | 63 | 7 | 24 | | |

A “cut off” for a negative result is a value of 13 ng/ml. This is calculated by determining the mean value for the normal subjects and adding 2 standard deviations to arrive at a 95% confidence level. The results indicate that two normal subjects gave a false positive result using the criteria stated above and one lung cancer patient gave a false negative result. For the sample given in Table VI the results are 95% accurate for negative results and 96.4% accurate for positive results.

### EXAMPLE 36

### Assaying For Collagen Fragment in Serum from Patients

Assays were performed according to the procedure set out in Example 32. The results obtained are presented Table V.

TABLE V

| | I | II | III | IV | V |
|---|---|---|---|---|---|
| Normal | 0.195 | 0.059 | 0.095 | 1.107 | 0.258 |
| Serum | | | | | |
| Ovarian | 0.305 | 0 | 1.188 | 0.056 | 0.313 |
| Cancer | | | | | |
| Adeno- | 0.123 | 0 | 1.237 | 0.116 | 1.181 |
| carcinoma | | | | | |
| Lung Cancer | 0.513 | 0.093 | 0.103 | 0.018 | 0.028 |
| Breast | 0.489 | 0 | 1.052 | 0.055 | 0.142 |
| Cancer | | | | | |

The above description of preferred embodiments of processes for the detection of cancer markers and associated methods are for illustrative purposes. Variations will be apparent to those skilled in the art. It will be apparent that the buffers and the concentrations used can be varied without changing the overall specification and preparation procedures. Also those skilled in the art are aware that concentrating protein solutions and removal of low molecular weight molecules from protein solutions can be achieved by many different methods, all of which will achieve the desired results.

What is claimed is:

1. A method for assaying for cancer in a mammal comprising:
attaching a trapping antibody to a surface, wherein the trapping antibody recognizes extracellular matrix (ECM) complexes;
contacting a biological fluid sample with the trapping antibody attached to the surface to thereby bind any ECM complex present in the biological fluid to the trapping antibody to form bound ECM complex;
preparing signaling antibody, wherein the signaling antibody recognizes ECM complex;
binding signaling antibodies to bound ECM complex; and
quantitating the amount of signaling antibody bound to the surface, wherein accumulation of signaling antibody on the surface, above a background accumulation, indicates a positive result for cancer.

2. A method for as recited in claim 1 wherein the antibodies are selected from the group consisting of monoclonal antibodies and polyclonal antibodies.

3. A method for as recited in claim 1 wherein the background accumulation is the accumulation obtained with the serum from a normal population.

4. A method as recited in claim 1 wherein the trapping antibody is prepared by a method comprising:
isolating an impure antibody composition;
contacting the impure antibody composition with ECM-complex-chromatography-medium to bind antibodies to the ECM complex; and
eluting the bound antibodies from the ECM-complex-chromatography-medium.

5. A method as recited in claim 1 wherein the signal antibody is prepared by a method comprising:
isolating an impure antibody composition;
contacting the impure antibody composition with ECM-complex-chromatography-medium to bind antibodies to the ECM complex;
eluting the bound antibodies from the ECM-complex-chromatography-medium; and
binding a label to the antibodies eluted from the ECM-complex-chromatography-medium.

6. The method as recited in claim 5 wherein the label is selected from the group consisting of enzyme labels, biotin labels, fluorescent labels and radioactive labels.

7. The method as recited in claim 1 wherein biological fluids which indicates a positive result for cancer are further tested to identify the type of cancera present by reacting the body fluids with anti-collagen antibodies.

8. A method of purifying trapping antibodies comprising:
providing an impure antibody composition;
contacting the impure antibody composition ECM-complex-chromatography-medium to bind antibodies to the ECM complex; and
eluting the bound antibodies from the ECM-complex-chromatography-medium.

9. The method as recited in claim 8 wherein the method further comprises contacting the antibodies eluted from the ECM-complex-chromatography-medium with normal-serum-chromatography-medium to bind contaminants to the normal-serum-chromatography-medium.

10. The method as recited in claim 8 wherein the method further comprises contacting the antibodies eluted from the ECM-complex-chromatography-medium with normal-plasma-chromatography-medium to bind contaminants to the normal-plasma-chromatography-medium.

11. The method as recited in claim 8 wherein the method further comprises contacting the antibodies eluted from the ECM-complex-chromatography-medium with type-IV-collagen-chromatography-medium to bind contaminants to the type-IV-collagen-chromatography-medium.

12. The method as recited in claim 8 wherein the method further comprises contacting the impure antibody composition with type-VI-collagen-chromatography-medium to bind antibodies to the type VI collagen.

13. A method as recited in claim 8 wherein the antibodies are selected from the group consisting of polyclonal antibodies and monoclonal antibodies.

14. A method of purifying signaling antibodies comprising:
providing an impure antibody composition;
contacting the impure antibody composition ECM-complex-chromatography-medium to bind antibodies to the ECM complex; and
eluting the bound antibodies from the ECM-complex-chromatography-medium.

15. The method as recited in claim 14 wherein the method further comprises contacting the antibodies eluted from the ECM-complex-chromatography-medium with normal-serum-chromatography-medium to bind contaminants to the normal-serum-chromatography-medium.

16. The method as recited in claim 14 wherein the method further comprises contacting the antibodies eluted from the ECM-complex-chromatography-medium with normal-plasma-chromatography-medium to bind contaminants to the normal-plasma-chromatography-medium.

17. The method as recited in claim 14 wherein the method further comprises contacting the antibodies eluted from the ECM-complex-chromatography-medium with type-IV-collagen-chromatography-medium to bind contaminants to the type-IV-collagen-chromatography-medium.

18. A method as recited in claim 14 wherein the antibodies are selected from the group consisting of polyclonal antibodies and monoclonal antibodies.

19. A method as recited in claim 14 wherein the method further comprises labeling the antibodies.

20. The method as recited in claim 19 wherein the label is selected from the group consisting of enzyme labels, biotin labels, fluorescent labels and radioactive labels.
